(19) United States
(12) Patent Application Publication
(10) Pub. No.: US 2010/0307515 A1
(43) Pub. Date: Dec. 9, 2010

(54) Foot Fulcrum

(76) Inventor: John Maurice Walsh, Bozeman, MT (US)

Correspondence Address: John Walsh, 28 F Shawnee Way, Bozeman, MT, 59715, US

(21) Appl. No.: 12/783,348

(22) Filed: May 19, 2010

Related U.S. Application Data
(60) Provisional application No. 61/184,425, filed on Jun. 5, 2009.

(51) Int. Cl. A61F 13/06

(52) U.S. Cl. 128/894

(57) ABSTRACT

The foot fulcrum orthotic includes a semi-compressible ring and attachment method to the foot. When worn around the midfoot, said semi-compressible ring forms a fulcrum to raise the ball of the foot up off the mattress and provides compression relief to the ball of the foot. This compression relief, when combined with other treatments, speeds the healing of metatarsalgia including Morton's neuroma, corns, calluses, and bunions. A plurality of ring styles are disclosed including continuous (FIGS. 7A, 7B, 7C), non-continuous (FIGS. 3, 4, 6A, 6B) with a gap for foot circumference size adjustment. A plurality of attachment methods are disclosed including single heel straps (FIGS. 7A, 7B, 7C), multiple heel straps (FIGS. 3, 4), socks (FIGS. 10A, 10B, 10C, 11A, 11B), and single-piece molded boot (FIGS. 9A, 9B).

### CROSS REFERENCE TO RELATED APPLICATIONS

This application claims the benefit of provisional patent application Ser. No. 61/184,425 filed 2009 Jun. 5 by the present inventor.

### FEDERALLY SPONSORED RESEARCH

Not applicable.

### SEQUENCE LISTING OR PROGRAM

Not applicable.

### BACKGROUND OF THE INVENTION

1. Field of the Invention

This invention is a device for treating metatarsalgia, or ailments associated with the ball of the foot. Such ailments include Morton's neuroma, calluses, corns, and bunions.

2. Description of the Related Art

Morton's neuroma is a painful swelling of the tissue around a nerve in the foot and is generally caused by pressure between two metatarsals on the common digital nerve between the bones. The most common location is between the third and fourth metatarsals. This pressure can come from a variety of sources such as inappropriate footwear, activities such as dance or tennis, and physical problems with the foot that affect gait.

A corn or callus is a thickening of the skin that occurs in an area of pressure. Corns appear as a horny thickening of the skin on the toes. This thickening appears as a cone shaped mass pointing down into the skin and can be very painful. Bunions are most commonly caused by inflammation and thickening of the bursa (fluid-filled sac in the connective tissue) and cause abnormal bone formation and misalignment of the toe. The location between the first toe and the metatarsal head is the area where bunions typically form. Symptoms include redness, swelling and a “bump” at the site.

The art related to treating metatarsalgia is old and crowded. Included herein are a few patents for prior art reference however, there are many similar patents related to the examples included. For example, the act of padding a corn or callus is well known to those skilled in the art. The comments regarding the referenced prior art are applicable across all the similar patents.

An example of treating corns, calluses and bunions is U.S. Pat. No. 5,497,789 (Zook, 1996) where he teaches a corn, bunion or blister pad incorporating a gel. His prior art discussion is detailed and goes back to padding patents from 1858. Padding is intended to minimize friction and pressure on the corn, callus or bunion. In most cased, the padding is worn over the afflicted area. The method of padding the area can be used while resting at night; however, the mattress can still apply pressure to the area. The padding just distributes the pressure so it is not focused on the afflicted area. The current invention involves removing the ball of the foot from the mattress surface entirely so there is nothing to come in contact with the area and no pressure to be distributed.

Prior art for the treatment of Morton's Neuroma pain has includes padding and strapping (taping), orthotics, shoe modifications, oral anti-inflammatory medications (NSAIDs), anti-inflammatory injectables (i.e., corticosteroids), injection of local injectables (i.e., peripheral nerve block), injection of sclerosing agents (i.e., Vitamin B-12, alcohol), analgesics, and physical therapy. If none of these treatments work, surgery to remove the neuroma is normally recommended.

The main focus of apparatus, as opposed to methods, for treating Morton's neuroma pain involves mitigating the pain while walking or wearing shoes. An example of this type of patent is U.S. Pat. No. 7,140,130 (Brooks, 2006) disclosing an insole with a neuroma pad. This insole is to be used while wearing shoes or sandals. The neuroma pad is intended to separate the metatarsals and relieve pressure on the neuroma. This type of treatment is not very comfortable to wear and is marginally useful to relieve some pain while walking. Brooks' invention offers no benefits when worn at night while resting in bed. There are a number of other patents that involve metatarsal pads incorporated directly into shoes. U.S. Pat. No. 4,250,886 (Riso et al., 1981) teaches a shoe with a built-in metatarsal pad. Again, these are a means to treat neuroma pain while walking, not resting.

The current invention is worn while resting or sleeping so some discussion regarding night orthoses is in order. Prior art for foot/ankle night orthoses is again a crowded field. Mainly, the focus has been on maintaining the foot and/or ankle in a fixed position while sleeping. A recent example of this is U.S. Pat. No. 7,182,743 (Slautterback et al., 2007) in which a foot splint is disclosed to maintain dorsiflexion when worn at night. U.S. Pat. No. 6,517,505 (Veldman, 2003) is a foot orthotic for treatment of foot pronation. Orthoses designed to treat plantar fascitis can be rigid as U.S. Pat. No. 7,182,743 (see above) or can take the form of a sock as U.S. Pat. No. 5,399,155 (Strassburg et al., 1995) where another method of maintaining dorsiflexion is disclosed. U.S. Pat. No. 6,468,239 (Mollura et al., 2002) teaches a device which attaches to the calf and keeps the foot elevated. While this device could possibly help a Morton's neuroma by keeping all pressure from the ball of the foot, a cursory look at the device shows no comparison with the current invention. Mollura's device attaches to the calf, using padding, straps, and a rigid frame. The current invention is much more simple and attaches to the foot, not the calf. The manufacturing costs of Mollura's invention would be substantially greater than the current invention. There are a number of other night orthoses to correct other types of problems, but none address a Morton's neuroma as the invention of this patent.

The current invention combines an innovative, non-obvious method of treating metatarsalgia pain using an apparatus worn while sleeping. The innovation of suspending the ball of the foot so no pressure is directed to the corn, callus, bunion, or neuroma in unique and non-obvious.

### BRIEF SUMMARY OF THE INVENTION

This invention relates to ailments of the ball of the foot that include but are not limited to Morton's neuroma, calluses, corns, and bunions and a device that when worn promotes and speeds the healing when used along with more traditional treatments.

The widest part of the foot is the ball of the foot. If one looks at the position the ball of the foot while a person is lying on one's side in bed, it can be easily seen that the ball of the foot receives the majority of the pressure created by the weight of the foot and ankle. This weight is being transferred from the ball of the foot to the surrounding mattress and causes compression on the metatarsal area.

This is typically the area of the neuroma, corn, callus, or bunion. In many situations, this foot and mattress alignment is consistent with sleeping in a prone position as well. The feet are splayed to the side putting even more pressure on the ball of the foot. A person sleeping exclusively on their back with their feet pointed straight up would not typically experience the benefits of this invention.

For Morton's neuroma, corns, calluses, and bunions, relief can be found in reducing the pressure at the ball of the foot. During waking hours, this can be achieved by adding padding to the affected area, adding insoles, or changing footwear or activities. During sleeping hours, the mattress can exert pressure in such as way as to exacerbate the problem. This invention addresses the problem of pressure on the ball of the foot when resting or sleeping.

The invention consists of a ring of semi-compressible material, such as closed cell foam, that when worn around the midfoot forms a fulcrum, which relieves pressure on the ball of the foot when the wearer is sleeping in bed or resting. The weight of the ankle and calf cause the ball of the foot to be raised off the mattress by the fulcrum. There are a plurality of methods for holding this ring in place.

Among the embodiments of this invention, a common thread is maintained: A semi-compressible ring or pad around the instep of the foot creating a fulcrum while leaving the ball of the foot to hang free in an uncompressed state while in bed. This is important because the compressed nerve or other ailment will experience relief for the duration of the rest/sleep period. The invention is not trying to address the specific cause of the metatarsalgia as with the prior art, such as poor footwear or incorrect foot biomechanics. It is assumed that the person with the painful ailment will be taking steps to mitigate these causes. The purpose of the invention is to facilitate the healing of the neuroma or other metatarsalgia while the person sleeps. In many cases, the relief from pain facilitated by wearing this invention will obviate the need for risky and expensive surgery.

### BRIEF DESCRIPTION OF THE DRAWINGS

FIG. 1 is a bottom view of a foot showing relevant bones and nerves;

FIG. 2A is a view of the pressure on a foot while resting in bed;

FIG. 2B is a view of the pressure on a foot while wearing an embodiment of the device and resting in bed;

FIG. 3 is a view of one embodiment of the device;

FIG. 4 is the right-hand view of one embodiment of the device shown as worn on the foot;

FIG. 5A is the left-hand view of one embodiment of the device shown as worn on the foot;

FIG. 5B is the left-hand view of one embodiment of the device shown as worn on the foot with an alternate attachment point for heel strap;

FIG. 6A is the bottom view of another embodiment of the device with the circumference adjustment located at the bottom of the device;

FIG. 6B is the top view of another embodiment of the device with the circumference adjustment located at the bottom of the device;

FIG. 7A is a view of another embodiment of the device shown with a continuous ring and a single long strap;

FIG. 7B is a left-hand view of another embodiment of the device shown with a continuous ring with the single long strap wrapped around a heel;

FIG. 7C is a right-hand view of another embodiment of the device shown with a continuous ring with the single long strap wrapped around a heel;

FIG. 8A is another embodiment of the device with a continuous ring and a short and long strap;

FIG. 8B is a left-hand view of another embodiment of the device shown with a continuous ring with the short and long strap fastened behind a heel;

FIG. 8C is a right-hand view of another embodiment of the device shown with a continuous ring with the short and long strap fastened behind a heel;

FIG. 9A is a view of another embodiment of the device shown as a single molded piece;

FIG. 9B is view of the single piece molded piece as worn on a foot;

FIGS. 10A, 10B, and 10C are views of another embodiment of the device showing a partial sock attached to a continuous ring; and

FIGS. 11A and 11B are views of the invention using two pads and a partial sock.

### REFERENCE NUMBERS

M1 First metatarsal
M2 Second metatarsal
M3 Third metatarsal
M4 Fourth metatarsal
M5 Fifth metatarsal
102 Medial plantar nerve
104 Lateral plantar nerve
105 Navicular bone
106 Cuneiform bones
107 Cuboid bone
108 Morton's neuroma
110 Phantom of invention shown around midfoot
202 Mattress
204 Pressure force from mattress on ball of the foot
206 Foot
208 Pressure force from mattress redirected to midfoot by invention
210 Ball of foot allowed to expand in uncompressed state
302 Hook and loop closure for circumference adjustment
303 Top strap for circumference adjustment
304 Right-hand heel strap
304A Right-hand heel strap loop closure
304B Right-hand heel strap hook closure
304C Right-hand heel strap loop alternate closure
304D Right-hand heel strap hook alternate closure
305 Left-hand heel strap
305A Left-hand heel strap loop closure
305B Left-hand heel strap hook closure
306 Fulcrum ring with circumference adjustment cutout on top
602 Bottom hook and loop closure for circumference adjustment
604 Bottom strap for circumference adjustment
606 Fulcrum ring with circumference adjustment cutout on bottom
702 Continuous fulcrum ring
704 Long single strap
704A First step in heel wrap of 704
704B Second step in heel wrap of 704
704C Third step in heel wrap of 704
704D Fourth step in heel wrap of 704
704E Fifth step in heel wrap of 704
704F Sixth step in heel wrap of 704
706A Loop closure for long strap
706B Hook closure for long strap
804 Medium strap with connector
806 Mated connector of medium and short strap
806A Connector for medium strap
806B Connector for short strap
808 Short strap with connector
902 Ankle part of molded boot
904 Ankle closure strap of molded boot
905 Hook and loop closure for ankle strap closure of molded boot
906 Foot closure strap of molded boot
907 Hook and loop closure for foot strap closure of molded boot
908 Foot part of molded boot
1002 Back of half sock
1004 Front of half sock extending from under fulcrum
1104A Foot pad
1104B Foot pad

### DETAILED DESCRIPTION

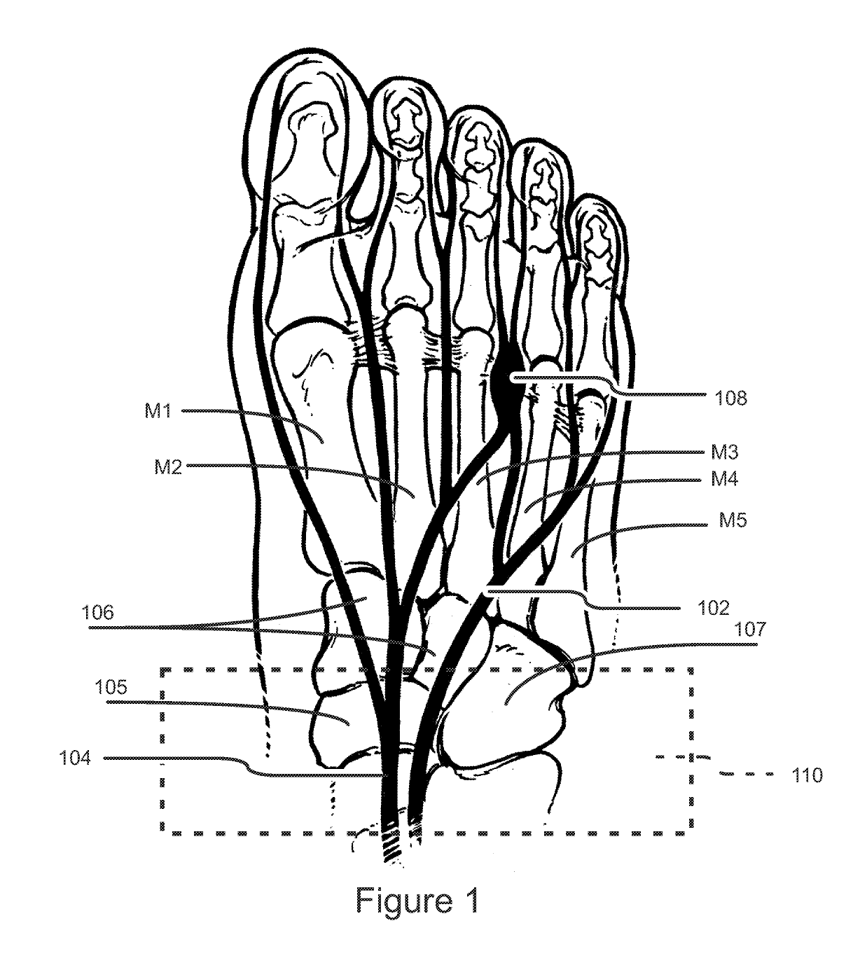

FIG. 1 of the drawings shows the bones and nerve structure of the foot. The figure shows the medial plantar nerve 102 and the lateral plantar nerve 104. M1-M5 designate the metatarsal bones. Also shown are the cuneiform bones 106 and navicular bone 105. The cuboid bone is show by item 108. 107 show the position of a neuroma. Phantom 110 shows the outline of the invention as worn on a person's foot.

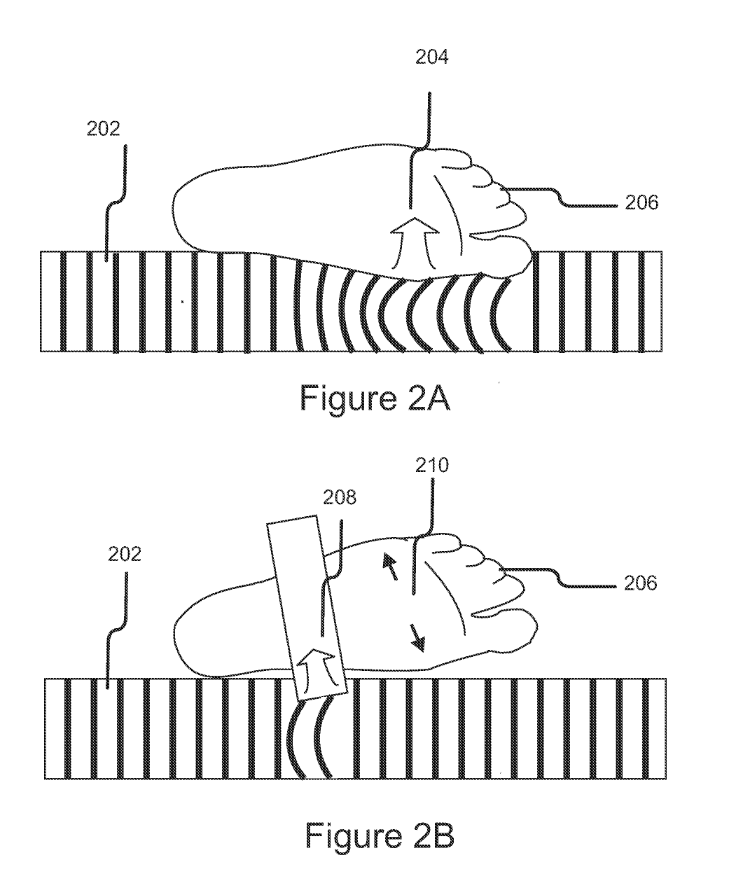

The benefits of the invention are shown in FIGS. 2A and 2B. FIG. 2A shows the foot resting on a mattress 202. As shown, the widest part of the foot is the ball. A force vector 204 shows the pressure from the mattress. With respect to corns, calluses, and bunions, this pressure is applied to the ailment and causes pain. With respect to Morton's neuroma, this pressure is directed into the ball of the foot and in fact, compresses the metatarsals together. Thus, the neuroma of FIG. 1, item 107, is additionally compressed when the person is resting or sleeping. The invention basically relieves the pressure on the ball while resting as shown in FIG. 2B.

FIG. 2B shows the foot at rest on a mattress 202, but in this figure, the person is wearing what amounts to a fulcrum 208 around their instep. This fulcrum shifts the pressure from the mattress 204 away from the ball and moves it to the midfoot, behind the metatarsals. The weight of the foot is carried on the cuneiform, navicular and cuboid bones. The shift, then allows the ball to be in an uncompressed state during rest as shown with the force vector 210. This is the key to the effectiveness of the invention.

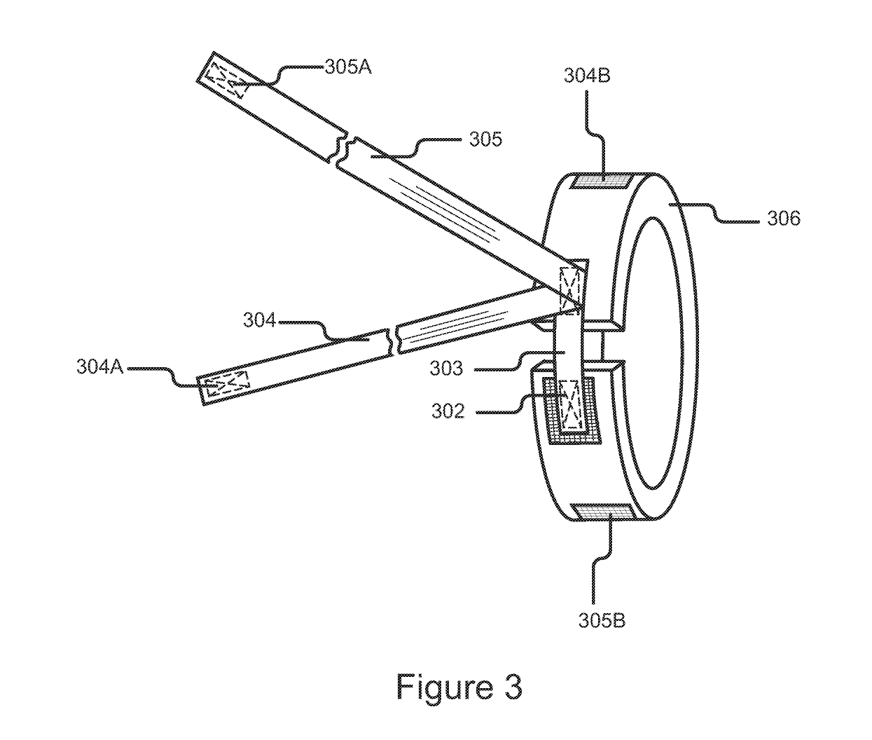

There are many ways to implement this invention and some of the embodiments of this invention are illustrated in FIGS. 3 through 11. These are only representational and are not intended to be restrictive in scope. In figures where a semi-compressible ring is mentioned, the ring can vary in diameter based on the foot size of the wearer. In general, the diameter can vary from 6 to 4 inches and the width of the ring can vary from 2 inches to 1 inch. The inner hole size will again vary based on the size of the wearer's foot. The size of the inner hole should leave a balance of material, basically the fulcrum aspect of the ring to be 2 inches to 1 inch per side.

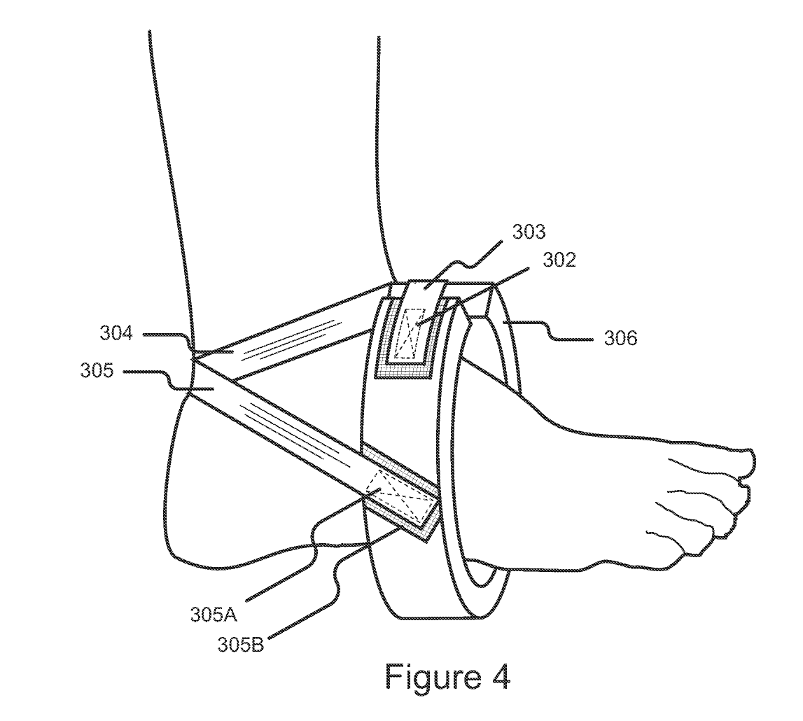

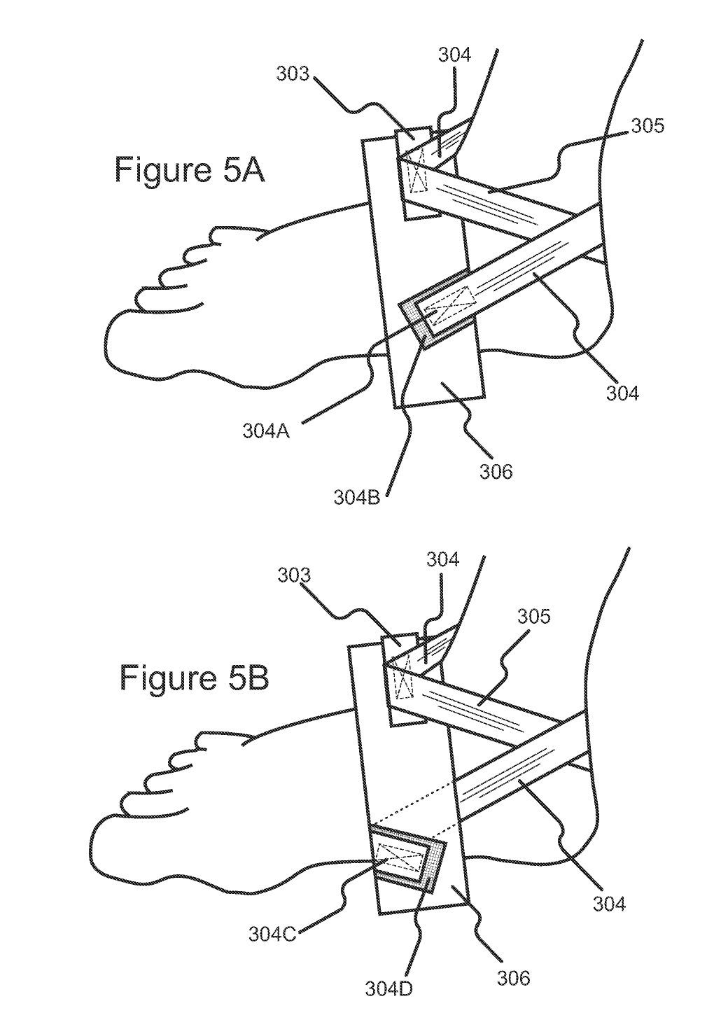

FIG. 3 shows one embodiment of the invention. The circular ring 306 creates the fulcrum effect when worn around the foot by placing the weight of the ankle and foot on the cuneiform, navicular, and the cuboid bones. This circular ring can be made of but is not limited to fabric covered semi-compressible foam. The fabric could be fleece or terry cloth for example and the ring could be made from closed call foam. The art of covering an item with cloth is well known in the art. Item 302 is an adjustable closure. It can be made from hook and loop material, a buckle, tied laces, or any simple method of closure. Item 303 is a strap that is used to join the two sides of the padded circular ring 306 together. The strap could be made either with some form of stretch material such as elastic or non-stretchable cloth tape webbing. This adjustment is not necessary and could be eliminated to save production costs. In this case, item 306 could be a continuous ring of material. The adjustment is provided as a means to accommodate the circumference differences of individual feet and to ensure a snug fit. The strap 305 and the closure made by the hook and loop 305A and 305B form the length adjustment to accommodate a variety of foot sizes. It should be noted that the closure for 305 is located on the opposite side of the ring 306. This is to allow the strap 305 to wrap around the ankle of the wearer and attach on the opposite side as shown in FIGS. 4, 5A and 5B. Strap 304 and hook and loop closures 304A and 304B offer an equivalent function for the opposite side of the foot. This method of straps and closures form a snug fit and prevent rotational and longitudinal shifting of the ring 306. One could easily imagine a version of this embodiment whereas the padding is slipped into two fabric pockets on the sides. One skilled in the art could also imagine a version with the padding being created from a bladder inflated with either air or some inert liquid or gel material.

FIG. 4 is a right side view of the embodiment shown in FIG. 3 and shows the device as worn on a foot. The items are as described in FIG. 3 with more detail shown of the straps 304 and 305. As shown, strap 305 wraps around the heel and attaches to item 306 on the side using hook and loop closure 305A and 305B. The straps 304 and 305 provide rotational and longitudinal stability of the padded circular element 306. The attachment point for the strap 304 is occluded in this figure and is shown in FIGS. 5A and 5B. Items 303 and 302 form an adjustable closure as described in FIG. 3. The key to the adjustment of elements 303, 304, and 305 and their associated hook and loop closures is to keep the padded circular element pulled back on the foot firmly to the heel. This location keeps the fulcrum effect on the cuneiform bones, the navicular bone, and the cuboid bone and allows the metatarsals to decompress while resting.

FIGS. 5A and 5B are left side views of the embodiment show in FIG. 3. FIG. 5A shows the straps 304 and 305 wrapping around opposite sides of the wearer's ankle and strap 304 attaching with the hook and loop closure 304A and 304B. An alternative to the 304A and 304B hook and loop closure is shown in FIG. 5B. In this figure, the strap 304 is looped under the ring 306 and folded over before being attached with the hook and loop closure 304C and 304D. This has the added benefit of more leverage being applied to ring 306 and less force trying to open the hook and loop closure 304C and 304D.

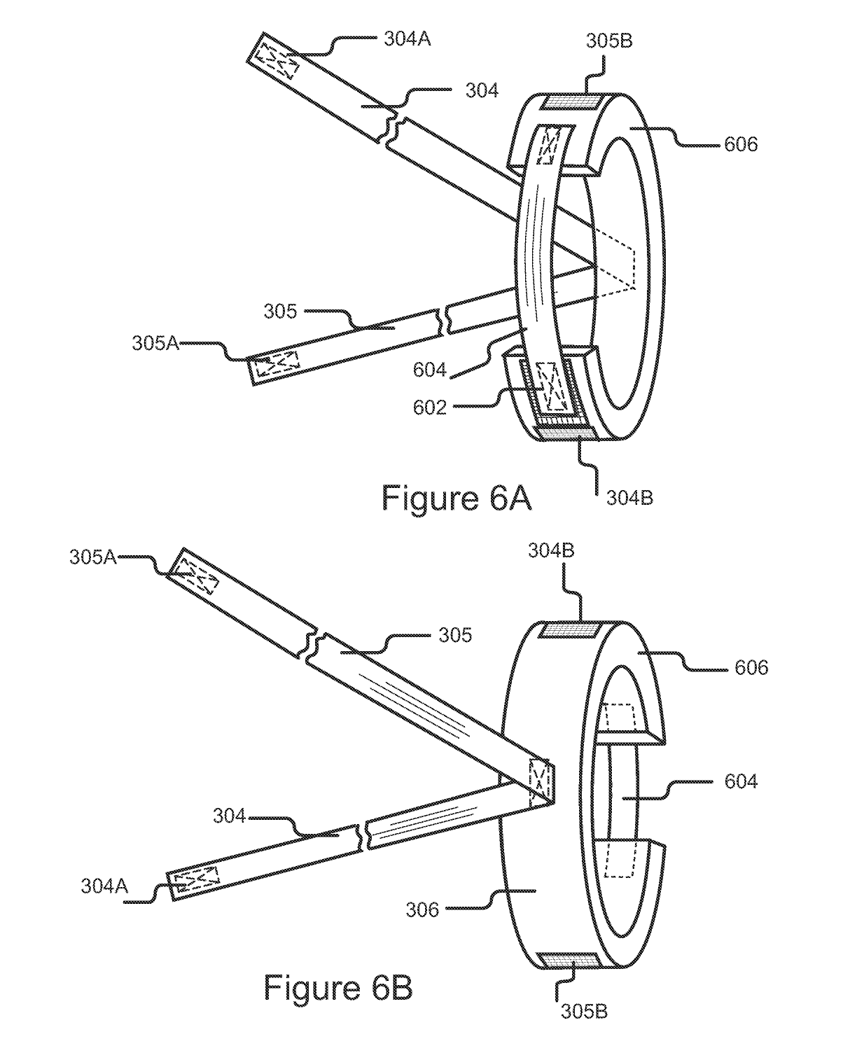

FIGS. 6A and 6B show an embodiment similar to FIG. 3. In this embodiment, the ring element 606 is closed at the bottom of the foot rather than the top. This creates a more flat area on the bottom of the foot and makes it easier for the wearer to walk with the invention. FIG. 6A is a bottom view with strap 604 and hook and loop closure 602 used to cinch the ring closed around the foot. Straps 304 with hook and loop closures 304A and 304B wrap around the foot and function in a similar manner as described in FIG. 3. The same is true of elements 305, 305A and 305B. FIG. 6B shows a top view with the circumference adjustment on the bottom and the straps 304 and 305 being attached to the top.

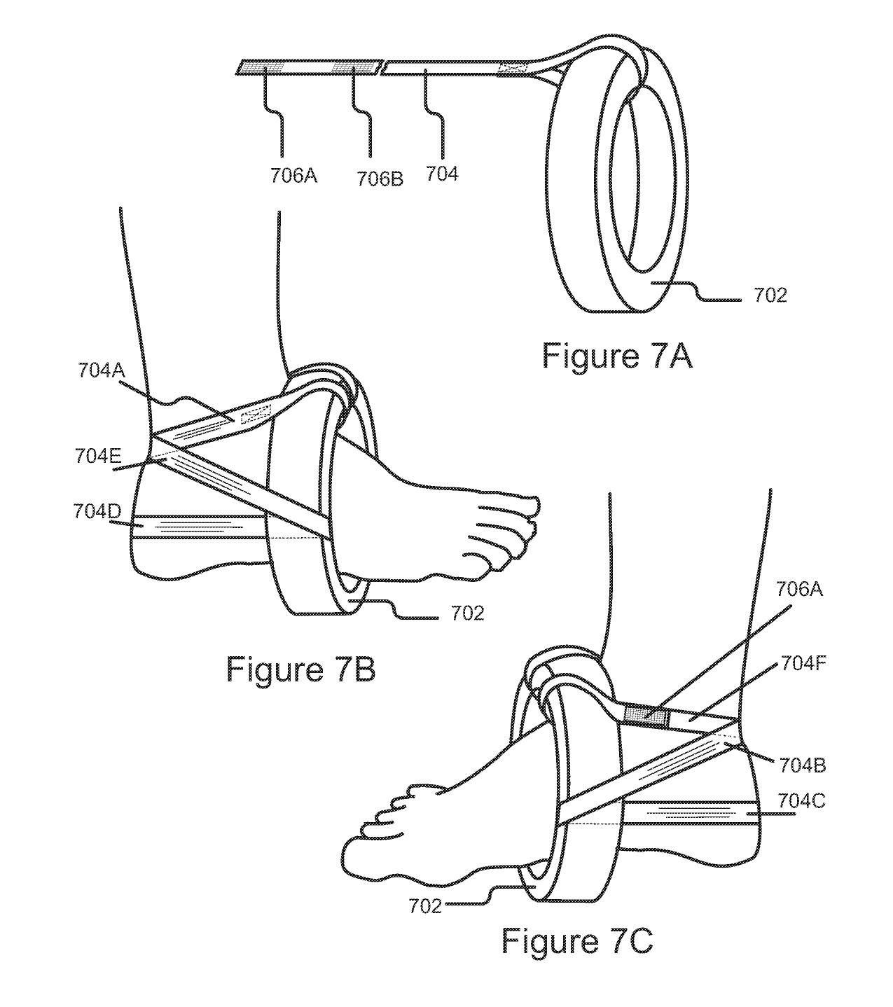

FIG. 7A shows another embodiment of the invention wherein the ring 702 is a continuous loop. A single strap 704 is either attached to the ring 702 or is looped over and attached to itself via sewn joint or similar method. The end of the strap 704 has both genders of hook and loop tape so it will adhere to itself when looped around the ring 702 in the final step of the attaching the device to wearer's foot.

FIGS. 7B and 7C show right hand and left hand sides of the foot while wrapping the strap 704. The first step is FIG. 7B. The strap is 704A. The strap is wrapped around the heel and becomes 704B in FIG. 7C. The next step is to wrap the strap under the ring and is shown as 704B turning into 704C as it passes under the ring. 704C wraps behind the heel and becomes item 704D in FIG. 7B. 704D then passes under the ring and wraps back to the heel as item 704E and in FIG. 7C becomes item 704F. 704F loops around the top of the ring and fastens back on to itself using the hook and loop closure 706A and 706B.

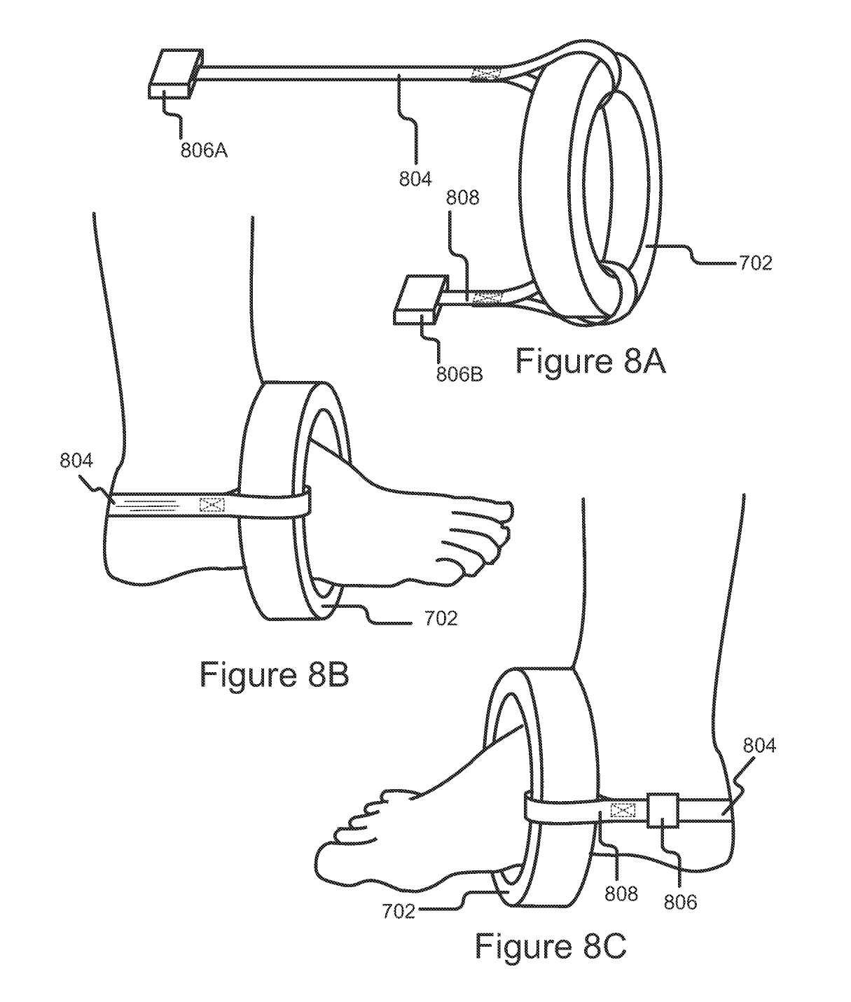

FIG. 8A is an additional embodiment of the invention showing another attachment method. In this embodiment, the ring'702 is the same as FIG. 7A, however there is a single strap going behind the heel. This strap is made from two pieces, items 804 and 808. Each end of the strap has the mating ends for the other end shown as 806A and 806B. FIG. 8B shows the right hand side of the embodiment and FIG. 8C shows the left hand side. In FIG. 8C, item 806 shows the mated connectors 806A and 806B. This connector can be hook and loop or any industry known equivalent such as a mating plastic connector, buttons, snaps or tied laces.

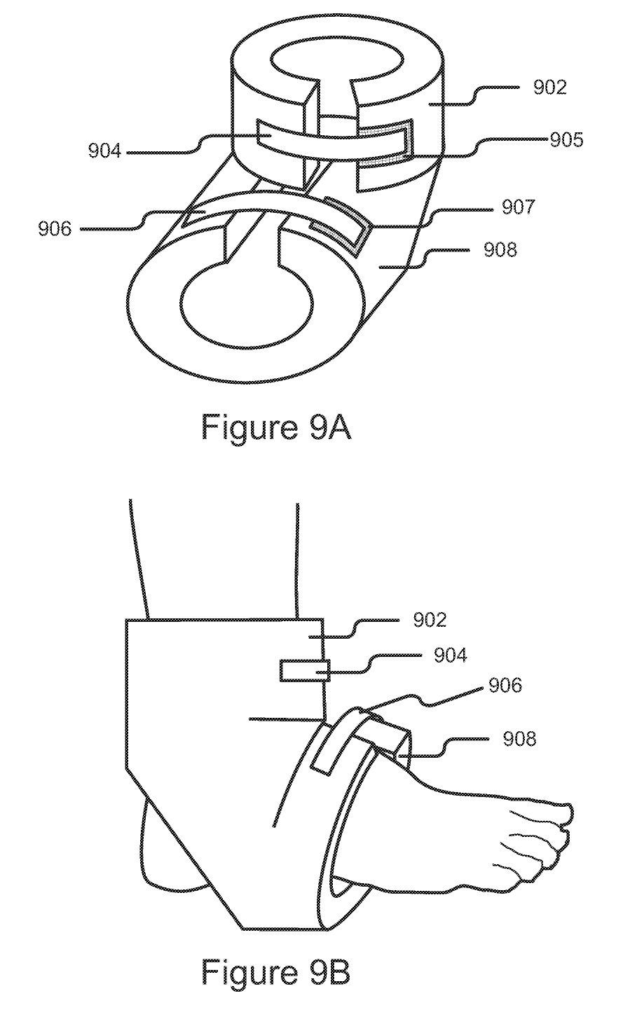

FIG. 9A is another embodiment of the invention. It consists of a form created from one or more pieces of semi-compressible foam, such as closed cell foam, that creates a boot form to surround the foot made with an ankle 902 and a shoe 908. The instep of the foot has a thicker band that forms the fulcrum. The ankle 902 is closed with a strap 904 and a hook and loop closure 905. The shoe element is closed with strap 906 and the hook and loop closure 907. This embodiment does away with the straps around the back of the foot for position stabilization and instead uses the boot form to hold the fulcrum ring in place. This embodiment also has the advantage of low production cost, as the material could be one step molded. One could also fabricate this embodiment with or without a fabric cover. If the base foam was left exposed, the wearer could pad the foot by using a standard sock. FIG. 9B shows a right hand side view of the embodiment on a foot.

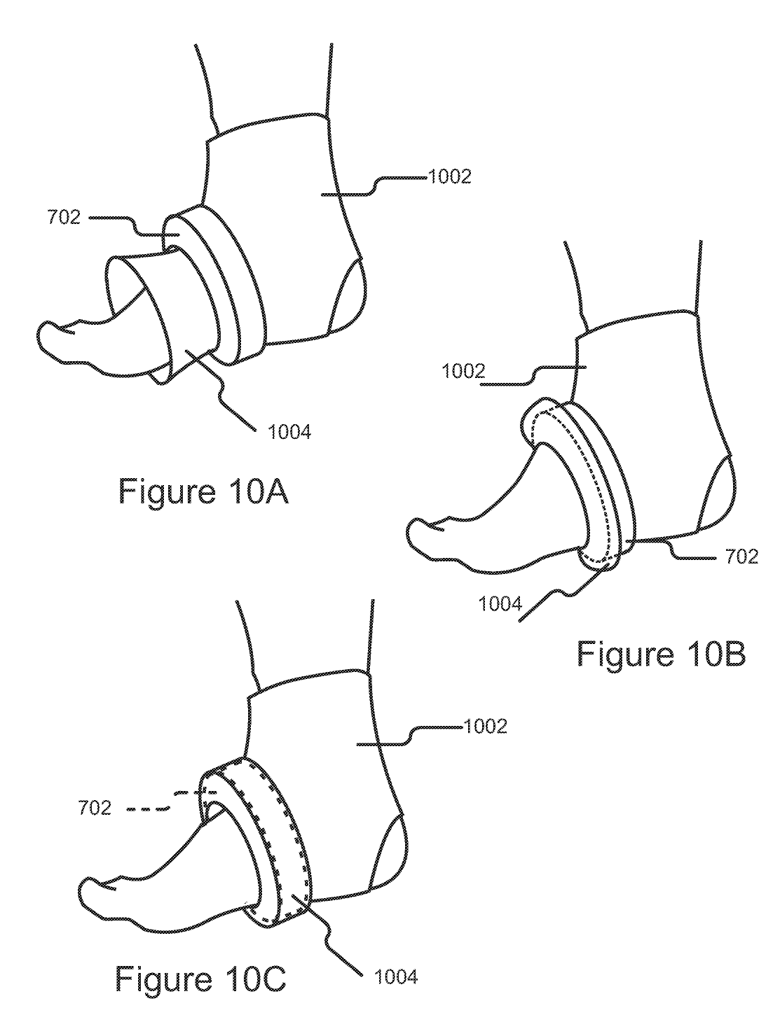

FIG. 10A is another embodiment shown with a half sock 1002 attached to a continuous semi-compressible ring 702. The sock could be made from standard sock material such as cotton, or it could be made of a material such as neoprene. The front of the sock 1004 is made long to fold around the semi-compressible ring and get attached back to itself and thus, stabilize the position of the ring. FIG. 10B shows the front of the sock 1004 being wrapped around the ring 702 and FIG. 10C shows the wrap completed and the front of the sock forming a pocket around the ring 702. This pocket secures the position of the ring 702.

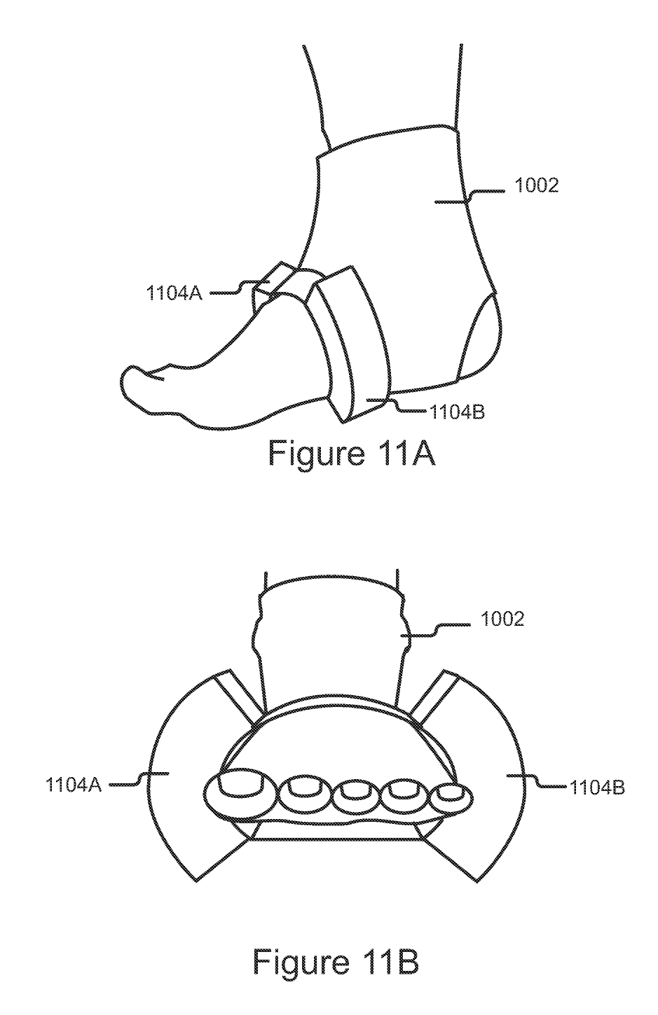

FIG. 11A is another embodiment shown with two pads 1104A and 1104B. These pads reside on the medial and lateral sides of the foot. The pads are held in place by the half sock 1002 to which they are attached. FIG. 11B is a frontal view of the embodiment shown in FIG. 11A. The thickness of the pad will vary based on the size of the wearer's foot but should range from 2 inches thick to 1 inch thick.

### CONCLUSION, RAMIFICATIONS, AND SCOPE

Accordingly the reader will see that, according to at least one embodiment of the invention, I have provided a unique, novel, and unobvious approach to dealing with metatarsalgia. This invention is very easy to use, has a minimum number of adjustments to be made for proper use. It can be safely worn for the duration of a nights rest without interfering with normal sleep. It is inexpensive to manufacture and provides an alternative to risky, painful, and oft times ineffectual surgery.

While the above description contains many specificities, these should not be construed as limitations on the scope on any embodiment, but as exemplifications of the presently preferred embodiments thereof. Many other ramifications and variations are possible within the teachings of the various embodiments. In many cases throughout the drawing descriptions, the term hook and loop closure is used. This is meant to be typical yet non-restrictive in nature. There are many ways to achieve the closures indicated such as snaps, buttons, and/or laces. Also, the semi-compressible ring could be made from closed cell foam or any other semi-compressible material such as bladders containing air, water, or gel. The semi-compressible ring can be continuous or have one or two gaps. In the case of a single gap, the gap can reside at the top or bottom of the foot. With two gaps, basically two pieces of padding are formed and they reside on the medial and lateral sides of the foot, over the midfoot bones. If material is used to cover the ring, or in the case of two pads mentioned above, the material can be made as pockets to contain the semi-compressible material so the ring or pads can be removed and the cover material can be laundered.

Thus the scope of the invention should be determined by the appended claims and their legal equivalents, not by the examples given.

1. A foot orthotic comprising:
a. a semi-compressible continuous ring worn around the midfoot and,
b. means of coupling said continuous ring to the foot,
whereby the pressure from the mattress to the ball of the foot is reduced at night relieving pain associated with metatarsalgia of the foot.

2. A foot orthotic comprising:
a. a semi-compressible non-continuous ring worn around the midfoot and,
b. means of coupling said continuous ring to the foot,
whereby the pressure from the mattress to the ball of the foot is reduced at night relieving pain associated with metatarsalgia of the foot.

3. A foot orthotic comprising:
a. a molded a boot of semi-compressible material and,
b. a cutout from the midfoot forward and,
c. means of adjusting boot to fit comfortably to foot,
whereby the pressure from the mattress to the ball of the foot is reduced at night relieving pain associated with metatarsalgia of the foot.

4. A foot orthotic in claim 1 compromising a means to stabilize the rotational position of said semi-compressible continuous ring.

5. A foot orthotic in claim 1 compromising a means to stabilize the toe to heel position of said semi-compressible continuous ring.

6. A foot orthotic in claim 2 compromising a means to stabilize the rotational position of said semi-compressible non-continuous ring.

7. A foot orthotic in claim 2 compromising a means to stabilize the toe to heel position of said semi-compressible non-continuous ring.

8. A foot orthotic in claim 2 compromising a means for foot circumference size adjustment.

9. A foot orthotic in claim 1 wherein the ring can be made of open or closed cell foam or any other semi-compressible material.

10. A foot orthotic in claim 2 wherein the ring can be made of open or closed cell foam or any other semi-compressible material.

11. A foot orthotic in claim 1 wherein said semi-compressible continuous ring can be covered in material to make it comfortable to wear next to the skin.

12. A foot orthotic in claim 2 wherein said semi-compressible non-continuous ring can be covered in material to make it comfortable to wear next to the skin.

13. A foot orthotic in claim 1 wherein said means of coupling said continuous ring to the foot is a sock with a sleeve midfoot encompassing said semi-compressible continuous ring.

14. A foot orthotic in claim 2 wherein said means of coupling said continuous ring to the foot is a sock with a sleeve midfoot encompassing said semi-compressible non-continuous ring.
